(19) United States
(12) Patent Application Publication
(10) Pub. No.: US 2009/0312408 A1
(43) Pub. Date: Dec. 17, 2009

(54) ISOFLAVAN AND ISOFLAVENE COMPOUNDS AND THEIR USE AS ANGIOGENESIS INHIBITORS

(76) Inventors: Herman Adlercreutz, Helsinki (FI); Carol Murphy, Ioannina (GR); Theodore Fotsis, Ioannina (GR); Nawaf Al-Maharik, Dundee (GB); Satu-Maarit Heinonen, Vantaa (FI)

Correspondence Address: BIRCH STEWART KOLASCH & BIRCH, PO BOX 747, FALLS CHURCH, VA, 22040-0747, US

(21) Appl. No.: 12/280,345

(22) PCT Filed: Feb. 22, 2007
(86) PCT No.: PCT/FI07/50092

§ 371 Date: Dec. 12, 2008

Related U.S. Application Data
(63) Continuation of application No. 11/361,229, filed on Feb. 24, 2006.

(30) Foreign Application Priority Data
Jun. 19, 2006 (FI) 20060601

(51) Int. Cl. A61K 31/35; C07D 311/00

(52) U.S. Cl. 514/456; 549/398

(57) ABSTRACT

Disclosed is the use of isoflavan and isoflav-3-ene compounds for the treatment of pathological conditions associated with or dependent on enhanced or abnormal angiogenesis in a mammal.

### FIELD OF INVENTION

The present invention is directed to a group of isoflavan and isoflavene (isoflav-3-ene) compounds and their use as angiogenesis inhibitors, especially in the treatment of pathological angiogenesis and angiogenic diseases, that is pathological conditions associated with or dependent on enhanced or abnormal angiogenesis, and in particular in the treatment of cancerous disease, such as in the suppression of tumor growth, in an individual, such as a mammal, typically in a human, in need of such treatment. The present invention also relates to a method of treatment of said pathological conditions using the said isoflavan and isoflavene compounds as active agents, as well as pharmaceutical compositions containing the said compounds.

### BACKGROUND OF THE INVENTION

Angiogenesis, the generation of capillaries, is virtually absent in the healthy adult organism and is restricted to a few conditions including wound healing and the formation of corpus luteum, endometrium and placenta. In contrast thereto, in certain pathological conditions angiogenesis is dramatically enhanced and is no longer self-limited, i.e. a result of well-balanced activity of angiogenesis inhibitors and stimulators. Pathological angiogenesis is seen during the development and progression of many diseases, such as in rheumatoid arthritis, psoriasis and diabetic retinopathy. Probably the clinically most important manifestation of pathological angiogenesis is that induced by solid tumors (Folkman, J. (1985) Adv. Cancer Res. 43, 175-203).

Estradiol metabolites, such as 2-methoxyestradiol, have been shown to exert an inhibitory effect on angiogenesis and on the proliferation of malignant cells. According to U.S. Pat. No. 5,643,900, 2-methoxyestradiol has been suggested for use in a method of treatment of pathological angiogenesis, especially for the treatment of solid tumors.

### SUMMARY OF THE INVENTION

The invention is based on the discovery that a group of compounds, which has the formula I,

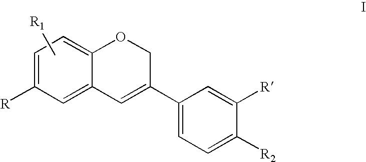

wherein R₁ and R₂ are independently hydrogen, —OR₃, —OCOR₄, —OCONR₅R₆, —OSO₂NR₅R₆ or —NH—CO—R₃,
and the group R₁ can be in the 7- or 8-position;
R and R′ are independently hydrogen or —OR₃;
R₃ is hydrogen or C₁₋₃ alkyl;
R₄ is C₁₋₃ alkyl; and
R₅ and R₆ are independently hydrogen or C₁₋₃ alkyl.
and the dotted line means an optional additional bond causing a double bond between carbons 3 and 4, with the proviso that one of the groups R and R₁ is an alkoxy group —OR₃, wherein R₃ is a C₁₋₃-alkyl group, and the other is a hydroxy group, and/or one of the groups R′ and R₂ is an alkoxy group —OR₃, wherein R₃ is a C₁₋₃-alkyl group, and the other is a hydroxy group, whereby such a hydroxy group R₁ and/or R₂ can be replaced by any of the other groups defined for R₁ and R₂ above, except hydrogen.

In the compound of the formula I the ring A denotes the fused benzene ring carrying the group R in the 6-position and R₁ in the 7- or 8-position of the ring system, and the ring B the phenyl ring carrying the group R′ in the 3′-position and R₂ in the 4′-position. According to the invention thus at least one of the rings A and B carries two substituents as defined. According to a preferred embodiment, in the compound of the formula I, there is a hydroxy group and an alkoxy group —OR₃, where R₃ is a C₁₋₃-alkyl group, preferably a methoxy group, preferably situated on adjoining carbon atoms, in only one of the rings A or B, preferably the ring A of the molecule. In such a case, the other ring, preferably ring B, is preferably either unsubstituted or substituted with a hydroxy group.

An object of the invention are also the above defined compounds and the use thereof in the form of one of their enantiomers, or in the form of the racemate.

The compounds of the formula I can find use as effective agents in a method of treatment of pathological conditions associated with or dependent on enhanced angiogenesis, in particular for, but not limited to the treatment of cancerous diseases, such as in the suppression of tumor growth, including that of solid tumors, in a mammal. The present invention also relates to a method of treatment using said angiogenesis inhibiting compounds of the formula I and a pharmaceutical composition containing the said angiogenesis inhibiting compounds of the formula I in combination with suitable pharmaceutically acceptable adjuvants.

### DETAILED DESCRIPTION OF THE INVENTION

In the formula I above, R₁ and R₂ have the meaning of hydrogen or hydroxy, or a group —OR₃, —OCOR₄, —OCONR₅R₆, —OSO₂NR₅R₆ or —NH—CO—R₃ that decreases the metabolism or increases the bioavailability of the compound. When a group R₁ and R₂ is defined as hydroxy, it is within the scope of the invention and this description that the said group can be replaced by a group as defined above, especially —OCOR₄, —OCONR₅R₆, —OSO₂NR₅R₆ or —NH—CO—R₃ that decreases the metabolism or increases the bioavailablity of the compound.

According to a preferred embodiment, there is at the most two, but usually only one alkoxy group —OR₃, where R₃ is a C₁₋₃-alkyl group, present in the compound of the formula I. In the case of two alkoxy groups present, these are preferably in separate rings in the molecule.

According to one embodiment, R₁ and R₂ have the meaning of —OR₃, wherein R₃ has the meaning defined, that is R₁ and R₂ have independently the meaning of hydroxy or C₁₋₃-alkoxy. R₁ is preferably in the 7-position of the benzene ring.

According to a further embodiment, R is hydroxy or C₁₋₃-alkoxy, typically when R′ is hydrogen.

According to a further embodiment R′ is hydroxy or C₁₋₃-alkoxy, typically when R is hydrogen.

A preferred group of compounds comprise those where there is a single bond between the carbon atoms 3 and 4 in the molecule.

According to a preferred embodiment, there is both a hydroxy group and an alkoxy group as defined present in adjacent positions in only one of the rings A or B. When the ring A is so substituted, one of R and R₁ is hydroxy and the other is alkoxy as defined. In such a case both of R′ and R₂ can be hydrogen, or one can be hydrogen and the other, preferably R₂, can be hydroxy. When the ring B is so substituted, one of R′ and R₂ is hydroxy and the other is alkoxy as defined. In such a case both of R and R₁ can be hydrogen, or one can be hydrogen and the other, typically R₁, can be hydroxy.

The C₁₋₃-alkoxy group is preferably the methoxy group.

The present invention specifically contemplates the use as defined of compounds of the formula I, wherein R is an alkoxy group —OR₃, where R₃ is C₁₋₃-alkyl, preferably methoxy. According to one embodiment, in such a compound R′ is hydrogen. According to a further embodiment, in such a compound both R₁ and R₂ are hydroxy. In a preferred embodiment R₁ is in the 7-position.

According to one embodiment, R is hydroxy. According to a further embodiment, in such a compound, R′ is hydrogen and R₁ is alkoxy —OR₃, wherein R₃ is C₁₋₃-alkyl, and preferably methoxy, preferably in the 7-position. In such a compound R₂ is advantageously hydroxy.

According to another embodiment, R is hydrogen. In such a compound, in a further embodiment, R₁ is preferably hydroxy. In such a compound one of R′ and R₂, preferably R′, is alkoxy —OR₃, wherein R₃ is C₁₋₃-alkyl, preferably methoxy, and the other is hydroxy. In this case R₁ can be in the 7- or 8-position.

A preferred compound in the group of compounds is 7,4′-dihydroxy-6-methoxyisoflavan.

The invention also contemplates the use of the compounds as defined in combination with or coupled to a biologically active molecule, for example a suitable targeting molecule, such as carrier molecules, e.g. peptides, or a suitable antibody, or other types of carriers which are capable of transporting the active agent to the desired target, such as a tumor, to exert its action.

Within the scope of the invention, the term “angiogenic disease” includes any pathological condition associated with or dependent on enhanced angiogenesis, that is a condition, which is directly or indirectly supported, sustained or aggravated by enhanced angiogenesis, i.e. abnormal angiogenesis. Such conditions include, but are not limited to cancerous diseases, such as solid tumors or tumor metastases, but can include also benign tumors, e.g. hemangiomas, abnormal wound healing, skin diseases such as psoriasis, ocular neovascular diseases, such as diabetic retinopathy and macular degeneration, and rheumatoid arthritis. The compounds can also be used for the treatment of leukemia and myeloma. The compounds can also be used in combination with other drugs, e.g. drugs used for chemotherapy of cancer and other diseases, including those listed above.

The invention is also directed to a pharmaceutical composition containing a compound as defined above, including a modified derivative thereof as defined, together with one or more pharmaceutically acceptable vehicles or other adjuvants,

According to a further aspect, the invention is directed to the use of the compounds as defined, including a modified derivative thereof as defined, for the preparation of a pharmaceutical composition for the treatment, including prophylaxis, of conditions associated with, or dependent on enhanced or abnormal angiogenesis.

In the context of this invention, an “effective” amount means a therapeutically or prophylactically effective amount, and such amounts can easily be established by the skilled person, taking into account the condition to be treated and the severity thereof, the age of the patient and the route of administration.

Any pharmaceutically acceptable adjuvant, including e.g. vehicles, carriers, fillers, excipients and additives for the manufacture of the composition may be used, and are as such well known to the person skilled in the art.

The compound of the formula I may be administered using any pharmaceutically acceptable form of administration. Suitable routes of administration include the oral route, such as in the form of capsules, tablets, granules, suspensions, the rectal route, such as in the form of suppositories, the parenteral route, such as by injection or infusion, or the topical route in the form of creams, lotions, or in the form of transdermal delivery systems or in the form of intraocular application, e.g. injection.

The amount of compound of the formula I to be included in the dosage form can be well determined by a person skilled in the art, and is dependant on the form of administration as well as the severity of the condition being treated. According to the invention, the compound of the formula I is administered to a subject in need thereof, typically in an amount of 0.1-500 mg per kg of body weight per day, preferably in an amount of 1-100 mg per kg body weight per day. Intraocular injections are not dosed in kg body weight, the amount being very small for such applications.

The compounds contemplated for use in the invention can be prepared using per se known methods, for example by reducing a compound having the formula

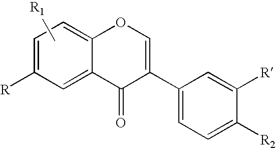

wherein the substituents have the meanings as defined above. The reduction can take place with hydrogen, e.g. on Pd/C in ethanol, to give a compound of the formula I having a single bond in the 3-position. Alternatively the reduction with hydrogen can be carried out over the corresponding 4-hydroxy compound, which subsequently can be dehydrated for example with an acid to the corresponding compound having a double bond in the 3-position. In this case it is possible to protect any hydroxy groups in the substituents e.g. by acetylation and to hydrogenate the so protected compound with hydrogen over Pd on basic alumina to the corresponding 4-hydroxy compound, which can be dehydrated e.g. with p-toluenesulfonic acid, whereafter the protecting groups can be removed, if desired, for example with a base. If desired, this compound with a double bond in the 3-position can be hydrogenated to a corresponding compound with a single bond in the 3-position.

A compound having one or more hydroxy groups can be converted to other groups according to known methods. Thus a hydroxyl group can be alkylated or acylated to an alkoxy or an ester group respectively by reacting the isoflavan or isoflav-3-ene compound with the corresponding alkyl or acyl halogenide, such as a chloride or a bromide, e.g. in dry DMF in the presence of potassium tert. butoxide. Sulfamoylation can be achieved for example by reacting the isoflavan or isoflav-3-ene compound with a corresponding N,N-dialkylamidochlorsulfonic acid in dry DMF in the presence of sodium hydride. The carbamate can be prepared for example by reacting the isoflavan or isoflav-3-ene compound with a corresponding N,N-dialkylcarbamoyl chloride for example in pyridine.

The starting compounds are well known isoflavone compounds found for example in legumes, such as soy.

Asymmetrically substituted compounds can be achieved by using the appropriate corresponding substituents in the starting materials used for making the starting compound, as defined above.

If necessary or desired, a hydroxy group in the starting material can be protected with a suitable protecting group, for example acylated, during a reaction and liberated after the reaction, in a known manner.

The following examples illustrate the invention, but are not intended to limit the scope thereof.

### Examples

### Synthesis of 7,4′-dihydroxy-6-methoxy-isoflavan

### 2,4,4′-Trihydroxy-5-methoxydeoxybenzoin

A mixture of 4-methoxyresorcinol (1.3 g, 9.29 mmol) and 4-hydroxyphenylacetic acid (1.27 g, 8.36 mmol) in BF₃.Et₂O (6.85 ml, 55.7 mmol) was heated at reflux temperature for 15 min under N₂. After cooling to room temperature, the dark solution was poured into saturated aqueous sodium acetate (100 ml) and extracted with ethyl acetate (3×50 ml). The combined ethyl acetate layer was washed with 10% aq. NaHCO₃ (50 ml) and water (100 ml), respectively, and dried with MgSO₄. The solvent was evaporated under reduced pressure, and the dark brown oil was subjected to chromatography (CH₂Cl₂:EtOAc=8:2) to give the title compound as a yellow solid (1.1 g, 48%), mp 157-159° C. ¹H NMR (300 MHz, DMSO-d₆) δ 12.38 (s, 1H, OH), 10.48 (br s, 1H, OH), 9.29 (br s, 1H, OH), 7.40 (s, 1H, H-6), 7.08 (d, J=8.6 Hz, 2H, H-2′,6′), 6.69 (d, J=8.6 Hz, 2H, H-3′,5′), 6.30 (s, 1H, H-3), 4.16 (s, 2H, CH₂), 3.76 (s, 1H, OCH₃); ¹³C NMR (75 MHz, DMSO-d₆) δ 202.2 (C—CO), 159.3 (C-4), 156.0 (C-4′), 155.4 (C-2), 141.0 (C-5), 130.4 (C-2′,6′), 125.3 (C-1′), 115.6 (C-3′,5′), 113.3 (C-6), 110.3 (C-1), 103.2 (C-2), 56.3 (C—OCH₃), 43.6 (C—CH₂). m/z=275 (M⁺1, 100%), 274 (16), 257 (9), 167 (18), 212; HRMS: C₁₅H₁₄O₅ requires 274.9841 found 274.0829.

### Glycitein

BF₃.Et₂O (1.6 ml, 13.13 mmol) was added to a solution of the deoxybenzoin obtained above (0.6 g, 2.19 mmol) in dry DMF (10 ml) under N₂. After 15 min, a solution of methanesulfonyl chloride (0.84 ml, 10.95 mmol) in dry DMF (2 ml) was slowly added. After heating at 70° C. for 5 h the reaction mixture was cooled to ambient temperature and poured into ice-cold saturated aq. sodium acetate (50 ml). The solid precipitate was filtered off and re-crystallized from 70% ethanol to give the title compound as a yellow solid (0.53 g, 85%), mp 336-338° C. ¹H NMR (300 MHz, DMSO-d₆) δ 10.63 (br s, 1H, 7-OH), 9.54 (br s, 1H, 4′-OH), 8.27 (s, 1H, H-2), 7.41 (s, 1H, H-5), 7.37 (d, J=8.7 Hz, 2H, H-2′,6′), 6.92 (s, 1H, H-8), 6.79 (d, J=8.7 Hz, 2H, H-3′,5′), 3.86 (s, 1H, OCH₃). MS (EI, 70 ev): m/z=284 (M⁺, 24%), 283 (100), 268 (15), 255 (20), 212 (41), 171 (15).

### 7,4′-Dihydroxy-6-methoxyisoflavan

Glycitein (0.5 g, 1.76 mmol) was reduced with H₂ over 10% Pd/C (0.25 g) in ethanol (50 ml) until no more H₂ was consumed. Pd/C was filtered off and the solvent was evaporated. The residue was purified by chromatography over silica gel (CH₂Cl₂:EtOAc=9:1) to give the title compound as a white solid (0.43 g, 90%) (from benzene), mp 159° C. ¹H NMR (300 MHz, CDCl₃) δ 7.11 (d, J=8.9 Hz, 2H, H-2′,6′), 6.81 (d, J=8.9 Hz, 2H, H-3′,5′), 6.55 (s, 1H, H-5), 6.48 (s, 1H, H-8), 5.54 (s, 1H, 7-OH), 4.85 (s, 1H, 4′-OH), 4.25 (ddd, J=1.8, 3.6, 10.5 Hz, 1H, H-2β), 3.92 (t, J=10.5 Hz, 1H, H-2α), 3.83 (s, 3H, 6-OCH₃), 3.11-3.24 (m, 1H, H-3β), 2.90-294 (m, 2H, H-4α, β); ¹³C NMR (75 MHz, CDCl₃) δ 154.5 (C-4′), 148.4 (C-8a), 144.8 (C-7), 140.9 (C-6), 133.75 (C-1′), 128.5 (C-2′,6′), 115.6 (C-2′,6′), 112.4 (C-4a), 111.5 (C-5), 103.1 (C-8), 70.9 (C-2), 56.5 (C-5), 38.0 (C-3), 32.2 (C-4). m/z=273 (M⁺1, 71%), 272 (100), 258 (48), 153 (18); HRMS: C₁₆H₁₆O₄ requires 272.1049 found 272.1059.

### Pharmacological Tests

In order to show the beneficial effects of the compound of the formula I, the following in vitro and in vivo tests were carried out on the compound 7,4′-dihydroxy-6-methoxy-isoflavan, in the following named F47.

### 1. Cell Culture

Human endothelial cells from umbilical vein (HUVEC) were plated on dishes pre-coated with rat collagen type I (Becton Dickinson Biosciences) and cultured in M199 medium supplemented with 20% fetal calf serum, FCS, 50 ng/ml endothelial cell growth supplement (ECGS, Sigma), heparin 10 μl (Sigma) and 1% penicillin-streptomycin. All media and sera for cell culture were purchased from Invitrogen and were endotoxin-free. F47 was tested for endotoxin content using the QCL1000 kit from BioWhittaker, Inc. Stock solutions of F47 were resuspended in DMSO/ethanol, 1/1 by volume, and added directly to the culture medium. Cells not receiving F47 were incubated in the corresponding volume of DMSO/ethanol.

### 2. Effect of F47 on Endothelial Cell Proliferation

### 2.1 Analysis by Cell Counting

BBCE cells were seeded (day 0) in 12-well tissue culture plates at a density of 1250 cells/cm² (5000 cells/ml/well) and the following day (day 1), wells received 5 μl of the compound dilutions to be tested and 2.5 ng/ml bFGF. This treatment was repeated after two days (day 3). On day 5 or 6, cells in duplicate wells were trypsinized and counted using a Coulter particle counter. Ten dilutions were tested for each compound.

### 2.2 Analysis by Ki67 Immunostaining

HUVECs (3×10⁴) were grown on cover slips and serum starved in a medium containing 5% FCS for 12 hr. Cells were induced with VEGF (50 ng/ml) in the presence or absence of F47 (10 μM) for 6 hr, fixed in 3.7% paraformaldehyde and processed for indirect immunofluorescence using an anti-Ki67 antibody. Cells were counterstained with Hoechst 33342. Proliferating cells (cells expressing the Ki67 antigen and simultaneously exhibiting intact, non-pyknotic nuclei) were recognized and counted using a Zeiss fluorescence microscope. Ki67 antigen is only expressed in active phases of the cell cycle, but not in G0 phase and the percentage of the Ki67-positive cells represents the proliferating population.

### 2.3 Results

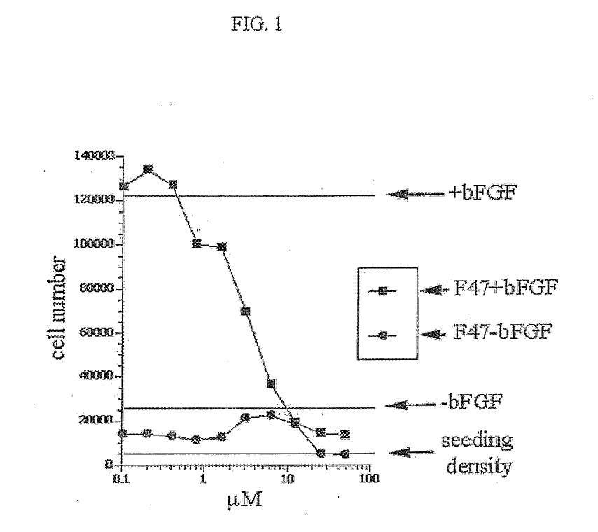

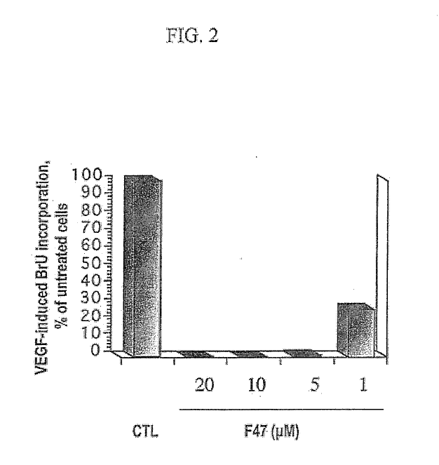

One of the main angiogenic endothelial cell responses is proliferation, in which the effect of F47 was examined. The effect of F47 on bFGF-induced endothelial cell proliferation was studied using cell counting, the results being shown in FIG. 1. In order to determine whether F47 could directly inhibit VEGF-induced proliferation, we have used Ki67 immunostaining because induction of cell proliferation by VEGF is week and cell counting is not suitable. F47 inhibited both bFGF- and VEGF-induced proliferation of endothelial cells with half-maximal concentrations of 3 and <1 μM, respectively. The effect of F47 on VEGF-induced proliferation of endothelial cells is shown in the FIG. 2.

### 3. Effect of F47 on Endothelial Cell Survival/Apoptosis

### 3.1 FACS Analysis

For analysis by flow cytometry, HUVECs were serum starved for 15 hr in medium containing 5% FCS and treated with VEGF (50 ng/ml) in the presence or absence of F47 (10 μM for the same period of time). At the end of the incubation time, floating and adherent cells were collected in ice-cold PBS, stained with propidium iodine using the CycleTEST PLUS DNA Reagent kit (Becton Dickinson Biosciences) and processed for flow cytometric analysis using a Becton Dickinson Fluorescence Activated Cell Scanner (FACS). The percentage of cells with a sub-G1 DNA content was considered as the cell population that had undergone apoptosis.

### 3.2 Results

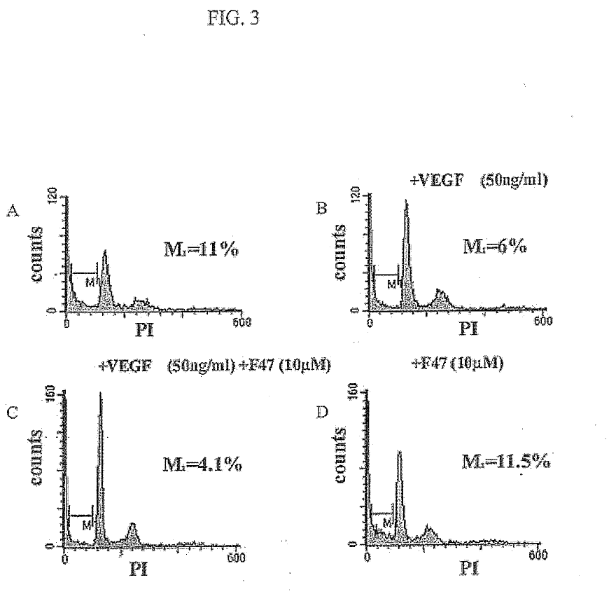

We investigated the effect of F47 on VEGF-induced survival of HUVECs, one important angiogenic endothelial cell response. Withdrawal of serum is well known to induce endothelial cell apoptosis, which is reversible upon VEGF addition. Therefore, we have examined the effect of F47 on VEGF-induced survival of HUVECs. Indeed, whereas serum-starved HUVECs (in 5% FCS) were apoptotic, being hypodiploid by FACS analysis (FIG. 3A), treatment of HUVECs with VEGF for 15 hr rescued almost 50% of the cells from apoptosis (FIG. 3B). However, treatment with F47 (10 μM) did not inhibit the VEGF-induced endothelial cell survival (FIG. 3C). F47 did not further increase the level of apoptosis of the serum-deprived HUVECs, excluding the possibility of toxic or apoptotic effects of F47 itself (FIG. 3D). Thus, F47 does not influence the effect of VEGF on survival of endothelial cells.

### 4. Effect of F47 on Endothelial Cell Invasion and Tube Formation

### 4.1 In Vitro Angiogenesis Assay

Collagen was solubilized from rat tail tendons essentially as described by Strom and Michalopoulos, Methods Enzymol 82: 544-555, 1982 and Dharmsathaphorn and Madara, Methods Enzymol. 192: 354-389, 1990. In order to prepare collagen gel, 8 volumes of a cold collagen solution from rat tail tendons (approximately 1.5 mg/ml) were quickly mixed with 1 volume of 10× minimal essential medium (MEM) without bicarbonate and 1 volume of sodium bicarbonate (11.76 mg/ml) on ice. F47 or DMSO was added at final concentration of 50 μM and the mixture was quickly dispensed into 2.00 cm²-tissue culture wells and it was polymerized at 37° C. for 10 min (Montesano et al., J. Cell Biol. 97: 1648-1652, 1983). Bovine Microvascular Endothelial (BME) cells were seeded onto collagen gel in 500 μl of D-MEM supplemented with 10% Donkey Calf Serum (DCS), 1% glutamine and 1% penicillin-streptomycin, containing F47 (50 μM) or DMSO (control sample). The cells were left at 37° C. for 90-120 min to be attached. Following that, the medium was removed and a second layer of 200 μl of collagen mixture, containing F47 (50 μM) or DMSO, was added on the top. Again, collagen was allowed to polymerize at 37° C. for 10 min. Finally, 500 μl of medium, containing either F47 (50 μM) or DMSO, was added on the top. The ultimate layer of the medium was changed every 2 days renewing thus F47 or DMSO. Pictures were taken after 7 days.

### 4.2. Results.

Identifying new molecular targets that block specific steps in endothelial cell morphogenesis may become crucial in efforts to inhibit pathological angiogenesis. Thus, we have investigated the effect of F47 on the tube formation of BME cells, using 3D extracellular matrix composed of rat-tail collagen. This matrix represents, together with fibrin, the major matrix environments in which angiogenic or vasculogenic events take place.

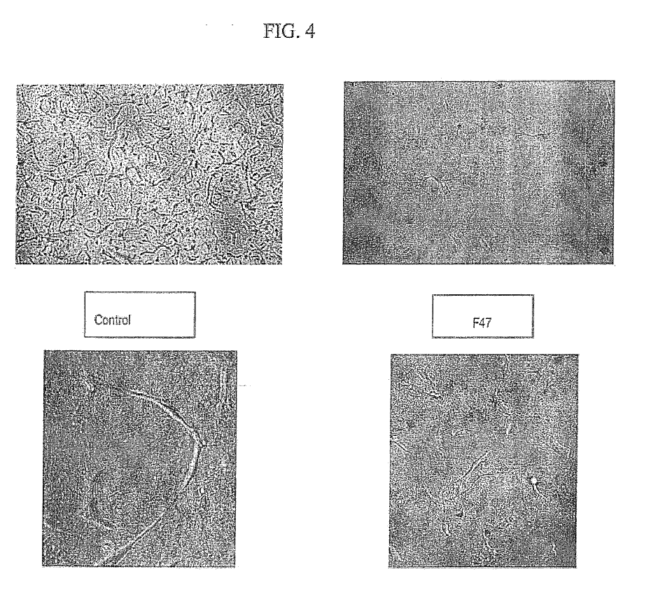

The results are shown in FIG. 4. The left hand pictures refer to the control and the right hand pictures to F47. F47 strongly inhibited in vitro angiogenesis in 3D collagen cultures. The photograph in the lower part of the figure was taken at a higher magnification.

### 5. Angiogenic/Antitumor Effect of F47 in Mice

The aim of the study was to evaluate the antiangiogenic/antitumor effect of F47 (5 μg/day) on a murine xenograft tumor model.

### 5.1 A-431 Murine Tumor Xenograft Model

To assess the in vivo antiangiogenic/antitumor activity of F47, female immunodeficient mice (5-8 week-old BALB/c nude mice, Charles River, Milan, Italy) were s.c. inoculated in the right flank with 10⁷ A-431 cells in a volume of 50 μl (Morbidelli et al., Clinic Cancer Res, 2003; 9(14): 5358-69). After 9 days, when tumors reached a volume of 170 mm³, animals were randomly assigned to 2 different experimental protocols (9-10 mice per group). Peritumor treatment with F47 (5 μg/day/mice) or vehicle started. The local peritumor treatment was performed at the dose of 5 μg/50 μl/mouse/day. The vehicle containing the same concentrations of solvents (1% ethanol+1% DMSO) was used as control. Daily treatment was performed for 10 consecutive days. Serial caliper measurements of perpendicular diameters were used to calculate tumor volume using the following formula: (shortest diameter×longest diameter×thickness of the tumor in mm). Data are reported as tumor volume in mm³. Experiments have been performed in accordance with the guidelines of the European Economic Community for animal care and welfare (EEC Law No. 86/609) and National Ethical Committee. Animals were observed daily for signs of cytotoxicity and were sacrified by CO₂ asphyxiation.

At day 10 animals were sacrificed and each tumor was immediately frozen in liquid nitrogen. Seven-μm-thick cryostat sections were stained with hematoxylin and eosin and adjacent sections were used for immunohistochemical staining with the anti-ED-B monoclonal antibody after fixation in absolute cold acetone.

### 5.2 Results

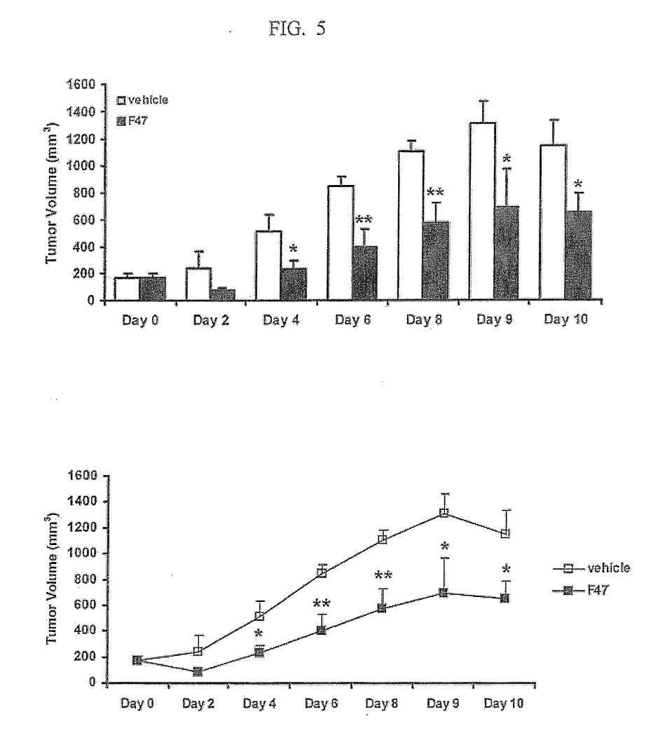

Treatment of A-431 tumors with F47 (5 μg/day/mice) reduced the growth of tumors as compared to the control group treated with vehicle. A reduction trend in tumor volume was seen in all the treated animals. However due to animal death, the tumor necrosis and ulceration starting from day 6 post-treatment, the numerical analysis was performed on 4 animals/group. Tumors in F47 treated mice were significantly smaller (approximately 50%, P<0.01 vs vehicle group at day 6 and 8) than in control mice beginning from day 2 (FIG. 5). Regarding survival, at day 8, survival of mice was 78% in the F47 group and 40% in the vehicle group.

FIG. 5 refers to the antitumor activity of F47 evaluated in nude mice inoculated with A-431 cells and treated after the onset of tumor growth (day 9 from inoculation, >150 mm³ tumor volume). Peri-tumor treatment with F47 (5 μg/mice/day) or vehicle continued for 10 days. Data are reported as tumor volume in mm³ (means±SEM of 4 animals/group).

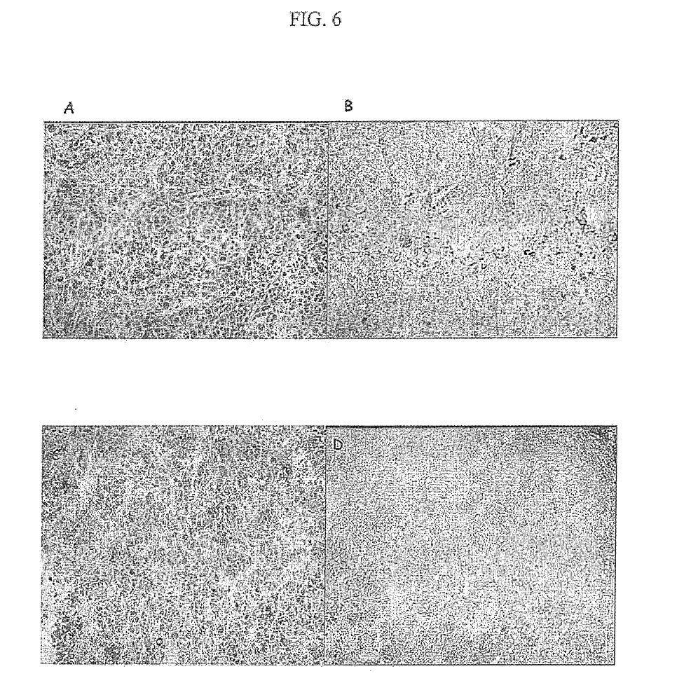

B-fibronectin (B-FN), the fibronectin (FN) isoform containing extradomain B (ED-B) accumulates around neovascular structures in aggressive tumors and other tissues undergoing angiogenesis and remodelling (Borsi et al., Blood. 2003; 102(13)-4384-92). The monoclonal anti-ED-B antibody against the ED-B domain in fibronectin (Pini et al., J. Biol. Chem. 1998; 273:21769-21776) evidenced the presence of tumor vasculature in tumors of the control group, which was absent in F47 treated tumors (FIG. 6).

In FIG. 6 the effect of 5 μg/day F47 (panels C-D) on tumor angiogenesis at day 10 was compared to vehicle treated group (panels A-B). The Figure gives representative pictures of tumor sections stained with hematoxylin and eosin (A,C) and with the antibody specific for B-FN (B,D). A positive signal (brown) was visible in microvessels and in the matrix undergoing remodelling due to tumor cell activation. Original magnification 20×.

1-28. (canceled)

29. A method of treating of pathological conditions associated with or dependent on enhanced or abnormal angiogenesis in a mammal in need of such treatment comprising administering an effective amount of a compound having the formula 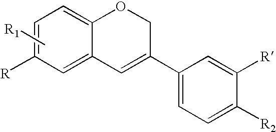
wherein R₁ and R₂ are independently hydrogen, —OR₃, —OCOR₄, —OCONR₅R₆, —OSO₂NR₅R₆ or —NH—CO—R₃
and the group R₁ can be in the 7- or 8-position;
R and R′ are independently hydrogen or —OR₃;
R₃ is hydrogen or C₁₋₃ alkyl;
R₄ is C₁₋₃ alkyl; and
R₅ and R₆ are independently hydrogen or C₁₋₃ alkyl.
and the dotted line means an optional additional bond causing a double bond between carbons 3 and 4, with the proviso that
i) one of the groups R and R₁ is an alkoxy group —OR₃, wherein R₃ is a C₁₋₃-alkylgroup, and the other is a hydroxy group, or one of the groups R′ and R₂ is an alkoxy group —OR₃, wherein R₃ is a C₁₋₃-alkyl group, and the other is a hydroxy group, or
ii) one of the groups R and R₁ is an alkoxy group —OR₃, wherein R₃ is a C₁₋₃-alkyl group, and the other is a hydroxy group, and one of the groups R′ and R₂ is an alkoxy group —OR₃, wherein R₃ is a C₁₋₃-alkyl group, and the other is a hydroxy group,
whereby such a hydroxy group R₁ and/or R₂ as defined in i) and ii) can be replaced by any of the other groups defined for R₁ and R₂ above, except hydrogen.

30. The method according to claim 29, wherein either one of the groups R and R₁ is an alkoxy group —OR₃, wherein R₃ is a C₁₋₃-alkyl group, and the other is a hydroxy group, or one of the groups R′ and R₂ is an alkoxy group —OR₃, wherein R₃ is a C₁₋₃-alkyl group, and the other is a hydroxy group.

31. The method according to claim 30, wherein, when one of the groups R and R₁ is an alkoxy group —OR₃, wherein R₃ is a C₁₋₃-alkyl group, and the other is a hydroxy group, R′ and R₂ are both hydrogen or one is hydrogen and the other is hydroxy, and wherein, when one of the groups R′ and R₂ is an alkoxy group —OR₃, wherein R₃ is a C₁₋₃-alkyl group, and the other is a hydroxy group, R and R₁ are both hydrogen or one is hydrogen and the other is hydroxy.

32. The method according to any one of claims 29 to 31, wherein, in the formula I, R is an alkoxy group —OR₃, where R₃ is a C₁₋₃-alkyl group.

33. The method according to claim 32, wherein R is methoxy.

34. The method according to claim 32, wherein R′ is hydrogen.

35. The method according to claim 29, wherein R₁ and R₂ are hydroxy.

36. The method according to claim 35, wherein R₁ is in the 7-position.

37. The method according to claim 29, wherein, in the formula I, R is hydroxy.

38. The method according to claim 37, wherein R₁ is alkoxy —OR₃, wherein R₃ is C₁₋₃-alkyl.

39. The method according to claim 38, wherein R₁ is methoxy.

40. The method according to claim 37 or 38, wherein R′ and/or R₂ are/is hydrogen.

41. The method according to any one of the claims 37 or 38, wherein R′ is hydrogen and R₂ is hydroxy.

42. The method according to claim 29, wherein one of R′ and R₂, is alkoxy —OR₃, wherein R₃ is C₁₋₃-alkyl, and the other is hydroxy.

43. The method according to claim 42, wherein R′ is methoxy.

44. The method according to claim 42, wherein R and/or R₁ are/is hydrogen.

45. The method according to claim 42, wherein R is hydrogen and R₁ is hydroxy.

46. The method according to claim 29, where there is a single bond between the carbons 3 and 4.

47. The method according to claim 29, wherein there is a double bond between the carbons 3 and 4.

48. The method according to claim 29, wherein the compound is 7,4′-dihydroxy-6-methoxy-isoflavan.

49. The method according to claim 29, wherein the compound is 7,4′-dihydroxy-6-methoxy-3,4-dehydro-isoflavan.

50. The method according to claim 29, wherein the condition to be treated is a cancerous disease.

51. The method according to claim 29, wherein the condition to be treated is a malignant solid tumor.

52. The method according to claim 29, comprising administering an amount of 0.1 to 500 mg/kg body weight/day.

53. The method according to claim 29, comprising using the compound in combination with or coupled to a targeting molecule, such as a biologically active molecule or carrier molecule, or other type of carrier capable of transporting the compound to the desired target.

54. The method according to claim 53, wherein the targeting molecule is an antibody or a peptide.

55. A compound having the formula 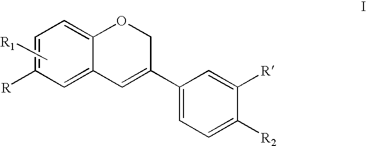
wherein R₁ and R₂ are independently hydrogen, —OR₃, —OCOR₄, —OCONR₅R₆, —OSO₂NR₅R₆ or —NH—CO—R₃,
and the group R₁ can be in the 7- or 8-position;
R and R′ are independently hydrogen or —OR₃;
R₃ is hydrogen or C₁₋₃ alkyl;
R₄ is C₁₋₃ alkyl; and
R₅ and R₆ are independently hydrogen or C₁₋₃ alkyl.
and the dotted line means an optional additional bond causing a double bond between carbons 3 and 4, with the proviso that
i) one of the groups R and R₁ is an alkoxy group —OR₃, wherein R₃ is a C₁₋₃-alkyl group, and the other is a hydroxy group, or one of the groups R₁ and R₂ is an alkoxy group —OR₃, wherein R₃ is a C₁₋₃-alkyl group, and the other is a hydroxy group, or
ii) one of the groups R and R₁ is an alkoxy group —OR₃, wherein R₃ is a C₁₋₃-alkyl group, and the other is a hydroxy group, and one of the groups R′ and R₂ is an alkoxy group —OR₃, wherein R₃ is a C₁₋₃-alkyl group, and the other is a hydroxy group,
whereby such a hydroxy group R₁ and/or R₂ as defined in i) and ii) can be replaced by any of the other groups defined for R₁ and R₂ above, except hydrogen,
for use as an agent for the treatment of pathological conditions associated with or dependent on enhanced or abnormal angiogenesis.

56. A compound according to claim 55 for use as an agent for the treatment of pathological conditions associated with or dependent on enhanced or abnormal angiogenesis.

57. The compound according to claim 55, for use as an anti-tumor agent.
